(19) United States
(12) Patent Application Publication
(10) Pub. No.: US 2017/0007516 A1
(43) Pub. Date: Jan. 12, 2017

(54) NAIL POLISH REMOVER

(71) Applicant: Elliona Pty Ltd., Cremorne NSW (AU)

(72) Inventors: Rochelle Rosemarie MERCADO, Cremome, NSW (AU); Fiona Lee REEVE, Dural, NSW (AU)

(21) Appl. No.: 15/125,865

(22) PCT Filed: Mar. 13, 2015
(86) PCT No.: PCT/AU2015/000138

§ 371 Date: Sep. 13, 2016

(30) Foreign Application Priority Data
Mar. 14, 2014 (AU) 2014900873

(51) Int. Cl. A61K 8/34; A61K 8/37; A61K 8/35; A61K 8/73; A61Q 3/04; A61K 8/04

(52) U.S. Cl. CPC A61K 8/345; A61Q 3/04; A61K 8/042; A61K 8/34; A61K 8/35; A61K 8/731; A61K 8/37

(57) ABSTRACT

A composition for removing nail polish from a surface, said composition comprising: (i) a solvent, said nail polish being at least partially soluble in said solvent; and (ii) a gelling agent, wherein the gelling agent forms a gel matrix, said solvent being included within said gel matrix.

### FIELD OF THE INVENTION

The present invention relates to nail polish removers and methods of removing nail polish.

### BACKGROUND

Nail polish removers are used to remove nail polish from fingernails, toenails and other surfaces. Some forms of nail polish, such as shellac and gel nail polish (polish that is set under UV or LED light), are harder to remove than other types. In order to remove such nail polishes using an existing nail polish remover, a greater exposure time of polish to the remover is generally required than for other, easier to remove nail polishes. Furthermore, existing non-acetone nail polish removers are generally not effective against such harder to remove nail polishes.

One method of removing harder nail polishes from nails is to soak the nails in an acetone bath. Soaking typically lasts for about 30 minutes, after which the nail polish is scraped from the nail. A problem with this method is that the skin of the finger is also in direct contact with acetone. Prolonged exposure to acetone can damage the skin. An alternate method is to place a cotton ball dipped in acetone on the nail and then wrap aluminium foil around both the nail and the cotton ball so as to hold the cotton ball in place. The cotton ball is left in place for about 30 minutes. This method also results in acetone contacting the skin, as evidenced by a white appearance of the finger when the foil wraps are removed. In some cases, prior to soaking in acetone, an electric nail buffer or drill is used to remove the top layers of nail polish prior to soaking in acetone. This can be damaging to both the nail and surrounding skin. Accordingly, there is a need for novel nail polish removers and novel methods for removing nail polish.

### OBJECT

It is the object of the present invention to substantially overcome or at least ameliorate one or more of the above disadvantages.

### SUMMARY

According to a first aspect of the invention, there is provided a composition for removing nail polish from a surface, said composition comprising: (i) a solvent, said nail polish being at least partially soluble in said solvent; and (ii) a gelling agent, wherein the gelling agent forms a gel matrix, said solvent being included within said gel matrix.

The following options may be used in combination with the above aspect, either individually or in any suitable combination.

The solvent of the composition may be acetone. The concentration of the solvent in the composition may be between 50 and 99% by weight of the total composition.

The gelling agent of the composition may be hydroxypropyl cellulose. The concentration of the gelling agent in the composition may be between 1.5 and 10% by weight of the total composition.

The composition may further comprise one or more emollients. The one or more emollients may be selected from the group consisting of cetyl alcohol, acetylated lanolin alcohol, caprylic/capric triglyceride, glycerine and combinations thereof. The concentration of the emollients in the composition may be between 1 and 20% by weight of the total composition.

The composition may further comprise a co-solvent. The co-solvent may be purified water or propylene glycol monomethyl ether. The concentration of the co-solvent in the composition may be between 1 and 45% by weight of the total composition.

The composition may be in the form of a weak gel. The composition may be in the form of a strong gel. Where the composition is in the form of a strong gel, the composition may have a first surface adapted to substantially conform to a fingernail or toenail.

According to a second aspect of the invention there is provided an article for removing nail polish from a human fingernail or toenail, said article comprising: (a) a substrate, said substrate having a first surface adapted to substantially conform to said fingernail or toenail; and (b) the composition of the first aspect of the invention; wherein said composition is disposed on said first surface of said substrate such that said composition is configured to contact said fingernail or toenail.

The following options may be used in combination with the above aspect, either individually or in any suitable combination.

The size and shape of the first surface of the substrate may substantially correspond to the size and shape of the fingernail or toenail. The first surface of the substrate may be approximately 1.5 cm wide and 2 cm long. The substrate may be substantially impermeable to the composition.

The article may further comprise a covering layer. The covering layer may be adapted to at least substantially cover the composition, such that the composition forms a layer between the first surface of the substrate and the covering layer. The covering layer may be substantially impermeable to the composition.

According to a third aspect of the invention there is provided a method of removing nail polish from a surface, the method comprising the steps of: (a) providing a composition of the first aspect of the invention or an article comprising said composition according to the second aspect of the invention; (b) contacting said composition with said nail polish; (c) allowing sufficient time for said solvent to diffuse from said gel matrix to at least partially dissolve said nail polish; and (d) removing said at least partially dissolved nail polish from said surface.

The following options may be used in combination with the above aspect, either individually or in any suitable combination.

The time in step (c) of the method may be less than 15 minutes. The time in step (c) of the method may be between 1 minute and 5 minutes.

The nail polish may be shellac or gel nail polish. The surface may be a human fingernail or human toenail.

### BRIEF DESCRIPTION OF FIGURES

Embodiments of the present invention will now be described, by way of an example only, with reference to the accompanying figures wherein:

FIG. 1 is a photograph of a fingernail coated with nail polish;

FIG. 2 is a photograph showing dispensation of the nail polish remover composition of Example 1 from a squeeze bottle;

FIG. 3 is a photograph showing the composition of Example 1 applied to the nail polish-coated fingernail of FIG. 1;

FIG. 4 is a photograph showing removal of nail polish from the fingernail of FIG. 3 using an orange wood stick; and

FIG. 5 is a photograph showing the fingernail of FIG. 4 after removal of the nail polish.

### DESCRIPTION OF EMBODIMENTS

A composition for removing nail polish from a surface, such as a human fingernail or toenail, is described herein. As used herein, the term “nail polish” refers generally to any cosmetic coating that may be applied to a fingernail or toenail and includes, for example, shellac and gel nail polish. The nail polish remover composition comprises a gelling agent and a solvent, the solvent being capable of at least partially dissolving the nail polish. The concentration of the gelling agent in the composition is such that the composition is in the form of a gel matrix, the solvent being included within the gel matrix. When in use, the solvent diffuses from the gel matrix so as to contact and dissolve the nail polish and allow its removal from the surface.

As the nail polish remover composition described herein is in the form of a gel, it remains in place on the surface once applied to the surface. Thus, for example, when removing nail polish from a fingernail or toenail, contact of the solvent with the skin surrounding the nail can be lessened, compared to existing methods, or avoided entirely. Buffing of the nail prior to application of the nail polish remover composition is not required, further reducing the risk of damage to the nail and the skin surrounding the nail (although buffing prior to application may further aid in removal of the nail polish). Also, the time required for removal of nail polish using the composition and method described herein is greatly reduced compared to existing compositions and methods.

The nail polish remover composition may be used for removing nail polish from any suitable surface. The surface may, for example, be a human fingernail or toenail, an artificial (e.g., acrylic or gel) fingernail or toenail, or any other suitable surface to which nail polish is adhered. In embodiments, where the nail polish is adhered to an artificial fingernail or toenail, which in turn is adhered to a human fingernail or toenail, the nail polish remover composition may be used to remove the nail polish without removing the artificial fingernail or toenail from the human fingernail or toenail. The composition may be used for removing any suitable nail polish from the surface. The composition may, for example, be used for removing shellac or gel nail polish from a surface. In particular embodiments, the composition may be used for removing shellac or gel nail polish from a human fingernail or human toenail.

The solvent is selected so as to be capable of at least partially dissolving the nail polish. The solvent may comprise acetone or it may consist essentially of acetone. Other suitable solvents that sufficiently dissolve nail polish may be included in the composition, either alone or in combination with acetone. For example, one or more solvents selected from methanol, ethanol, ethyl acetate and combinations thereof, may be suitable for inclusion in the composition, either alone or in combination with acetone. The concentration of the solvent in the composition may be any suitable concentration sufficient for dissolving the nail polish. In embodiments, the concentration of the solvent in the composition may be between 50 and 99%, between 60 and 98%, between 70 and 95%, between 80 and 92%, or between 85 and 90% by weight of the total composition.

The gelling agent is selected so as to be capable of forming a gel matrix, the solvent being included within the gel matrix. As the composition is in the form of a gel matrix it will stay in place on a surface once applied, where existing liquid nail polish removers would run off the surface. The gel matrix may be either a strong gel or a weak gel. Strong gels are viscoelastic solids that rupture when sufficient force is applied, whereas weak gels are structured fluids that flow when sufficient force is applied. The gelling agent may have self-adhesive properties, such as tackiness, that assist in holding the composition in place after application to a nail polish-coated surface or to a substrate. The gelling agent may comprise hydroxypropyl cellulose or may consist essentially of hydroxypropyl cellulose. Other suitable gelling agents capable of forming a gel matrix with the solvent included therein may also be used alone or in combination with hydroxypropyl cellulose. The concentration of the gelling agent in the composition may be any suitable concentration sufficient for the composition to be in the form of a gel matrix. The concentration of the gelling agent in the composition may be between 1.0 and 5.0%, between 2.0 and 3.0%, or between 2.25 and 2.75% by weight of the total composition.

The composition may include any additional components suitable for inclusion in a nail polish remover composition. For example, the composition may comprise one or more co-solvents. Any suitable co-solvent may be included in the composition. In particular embodiments, the composition comprises purified water or propylene glycol monomethyl ether as a co-solvent. The concentration of the co-solvent in the composition may be up to 45%, up to 35%, up to 25%, up to 15%, or up to 5% by weight of the total composition. In embodiments, the concentration of the co-solvent in the composition may be between 1 and 45%, between 2 and 20%, between 5 and 15%, between 7 and 10%, between 25 and 45%, or between 30 and 40% by weight of the total composition. The composition may comprise one or more emollients. Any suitable emollients may be used in the composition. In particular embodiments, the composition comprises one or more emollients selected from the group consisting of cetyl alcohol, acetylated lanolin alcohol, caprylic/capric triglyceride, glycerine and combinations thereof. The composition may comprise Acetulan™ (Lubrizol), a commercially available emollient composition. The concentration of the one or more emollients in the composition may be up to 20%, up to 15%, up to 10%, up to 7.5%, up to 5%, or up to 2.5% by weight of the total composition. In embodiments, the concentration of the one or more emollients in the composition may be between 1 and 20%, between 1.5 and 15%, or between 2 and 10% by weight of the total composition.

Where the nail polish remover composition is in the form of a weak gel, the composition is able to flow under sufficient applied force. Thus, where the composition is in the form of a weak gel, the composition may be readily spread over a surface to which is has been applied. It may also be stored in and dispensed from a squeezable bottle, such as a polyethylene terephthalate or high-density polyethylene bottle.

Where the nail polish remover composition is in the form of a strong gel, the composition is a viscoelastic solid. Thus, the composition will hold its shape under normal storage conditions. If deformed, the composition will return to its original shape once the deforming force is removed, unless sufficient force is applied to cause rupture of the gel matrix. Thus, where the composition is in the form of a strong gel, it may be supplied in a solid unit form. In embodiments, the unit form of the composition has a first surface that is configured to substantially conform to a surface to which nail polish is adhered. For example, where the composition is for removing nail polish adhered to a human fingernail or toenail, or for removing nail polish adhered to an artificial fingernail or toenail, the first surface of the unit form is adapted to substantially conform to the fingernail or toenail. The first surface of the unit form may be sized and shaped to substantially cover the fingernail or toenail. Thus, the shape of the first surface of the unit form according to embodiments generally corresponds to that of a human fingernail or toenail or artificial fingernail or toenail. In embodiments, the size of the first surface of the unit form is approximately 1.5 cm wide, 2 cm long and 3 mm thick. However, the size of the first surface of the unit form will depend on the size of the surface for which the article is adapted. Other sizes that at least substantially cover a fingernail or toenail are envisaged.

In embodiments of the invention, the nail polish remover composition described herein is disposed on a substrate. Thus, there is also disclosed herein an article for removing nail polish adhered to a surface, the article comprising a substrate having the composition disposed thereon. Where the composition is a strong gel it is disposed on the substrate such that, in use, the composition is contactable with the nail polish-coated surface after removal of the substrate. That is, the surface of the composition in contact with the substrate is rendered contactable with the nail polish-coated surface after removal of the substrate. Where the composition is a weak gel it is disposed on the substrate such that, in use, the composition is contactable with the nail polish-coated surface without requiring the substrate to be removed.

The substrate has a first surface, on which the composition is disposed. The first surface is configured to substantially conform to a surface to which nail polish is adhered. For example, where the article is for removing nail polish from a human fingernail or toenail, or for removing nail polish adhered to an artificial fingernail or toenail, the first surface is adapted to substantially conform to the fingernail or toenail. The first surface may be sized and shaped to substantially cover the fingernail or toenail. Thus, the shape of the substrate according to embodiments of the invention generally corresponds to that of a human fingernail or toenail or artificial fingernail or toenail. In embodiments, the size of the substrate is approximately 1.5 cm wide, 2 cm long and 3 mm thick. However, the size of the substrate will depend on the size of the surface for which the article is adapted. Other sizes that at least substantially cover a fingernail or toenail are envisaged.

The substrate may be constructed of one or more layers of any suitable natural or synthetic material. The material may be substantially permeable, partially permeable or substantially impermeable to the composition. The material may be a woven or non-woven fabric or a flexible plastic sheet. Suitable materials may include polypropylene and natural fibres, such as bamboo. In embodiments, the substrate comprises at least two layers, at least one layer being substantially or partially permeable to the composition and at least one other layer being substantially impermeable to the composition. The substantially or partially permeable layer absorbs the composition, thereby holding it in place on the substrate, while the impermeable layer provides a barrier against leakage of the composition.

The article optionally includes a covering layer that is adapted to at least substantially cover the composition on the substrate, such that the composition forms a layer between the substrate and covering layer. The covering layer is removable so as to expose the composition, thus allowing the composition to contact the nail polish when using the article. The covering layer may be any suitable natural or synthetic material that is substantially insoluble in the solvent and allows for temporary engagement with the composition, but allows for the covering layer to be removed from the composition. The temporary engagement of the covering layer protects any self-adhesive nature of the composition to ensure that the self-adhesive property is not diminished or reduced before application. The covering layer material is substantially impermeable to the composition so as to provide a barrier against leakage of the composition. Thus, in embodiments, the article comprises a composition disposed on a substrate, the substrate comprising at least one layer that is substantially impermeable to the composition, and a covering layer that is also substantially impermeable to the composition, thereby providing a composition that is encased between the substrate and covering layer until the covering layer is removed. In embodiments, the covering layer is a flexible plastic sheet. For example, where the solvent is acetone, the covering layer may be a polypropylene sheet.

In using the nail polish remover composition described herein, the composition is contacted with the nail polish to be removed. The solvent of the composition then diffuses from the gel matrix and contacts the nail polish. Sufficient time is allowed for the solvent to diffuse from the gel matrix to at least partially dissolve the nail polish. Partial dissolution of the nail polish may be sufficient to loosen the nail polish from the surface, thereby allowing its removal from the surface. Once sufficiently dissolved, the composition and the at least partially dissolved nail polish are removed from the surface by, for example, wiping or scraping with a suitable implement. Thus, there is provided herein a method for removing nail polish from a surface.

The required time of exposure of the composition to the nail depends on the solubility of the nail polish in the solvent and the thickness of the nail polish. Greater exposure times are required where the nail polish is less soluble in the solvent and/or where the nail polish layer is thicker. In embodiments, an exposure time of less than 15 minutes is required to sufficiently dissolve the nail polish to allow its removal from the surface. In particular embodiments, less than 10 minutes, 5 minutes, 4 minutes, 3 minutes or 2 minutes is required. In particular embodiments, the required exposure time is between 1 and 5 minutes, between 1 and 4 minutes or between 1 and 2 minutes.

A description of one method of removing nail polish from a fingernail or toenail using the composition described herein is as follows. The hands and/or feet of the recipient may be washed prior to commencing application of the composition. An aloe vera or sorbolene cream is optionally placed on the skin around the nail and cuticle to act as a barrier and protect the skin against the solvent. The composition is then placed directly on the nail polish, for example, by dispensing the composition from a squeeze bottle, so as to contact and at least partially coat the nail polish to be removed. Care is taken to avoid contacting the composition with the surrounding skin. The composition is allowed to remain in place until the solvent has diffused from the gel matrix and sufficiently dissolved the nail polish to allow its removal. After such time the composition and nail polish are scraped off the nail using a wooden cuticle tool. Any remaining aloe vera or sorbolene cream on the finger and/or toe may be massaged into the nail.

### EXAMPLES

Nail polish remover compositions were prepared using the components in Table 1 below. The component concentrations given in Table 1 are relative to the total weight of the composition.

TABLE 1
Nail polish remover compositions

| | Concentration (% wt.) | | | | |
|---|---|---|---|---|---|
| Component | Ex. 1 | Ex. 2 | Ex. 3 | Ex. 4 | Ex. 5 |
| Acetone | 88.5 | 84.0 | — | — | — |
| Ethanol | — | — | 91.5 | 58.0 | 52.5 |
| Ethyl Acetate | — | — | — | — | — |
| Purified Water | 7.0 | 10.0 | — | — | — |
| Propylene Glycol Monomethyl Ether | — | — | — | 30.0 | 40.0 |
| Acetulan ™ | 2.0 | 4.0 | — | — | — |
| Caprylic/Capric Triglyceride | — | — | 4.0 | 10.0 | 5.0 |
| Glycerine | — | — | 2.0 | — | — |
| Hydroxypropyl cellulose | 2.5 | 2.0 | 2.5 | 2.0 | 2.5 |

### Example 1

In preparing the nail polish remover composition of Example 1, the acetone and purified water were combined and then mixed until uniform. The Acetulan™ was then added and the combined components mixed until uniform. The hydroxypropyl cellulose (Klucel HF Pharm) was then sprinkled into the batch while mixing. The mixing was continued until the hydroxypropyl cellulose was completely dispersed and the batch composition thickened to form a gel.

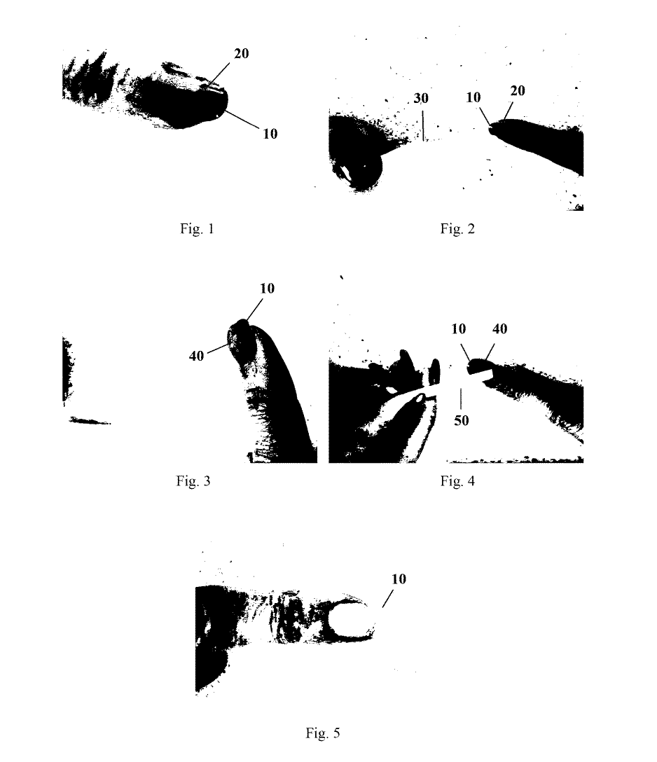

The nail polish remover composition of Example 1 was tested on a fingernail (10) to which shellac (20) was adhered (FIG. 1(a)). From a squeeze bottle (30), a small amount of the composition was applied to the nail polish-coated fingernail (FIG. 1(b)). The amount of composition (40) applied to the fingernail was sufficient to completely coat the nail polish layer (FIG. 1(c)). Once dissolution of the nail polish commenced, the nail polish was removed with an orange wood stick (60) by lifting the partially dissolved nail polish from the fingernail (FIG. 1(d)).

Dissolution of the nail polish began immediately upon application of the composition of Example 1 to the nail polish. At 1 minute and 40 seconds after application of the composition most of the nail polish was removed from the fingernail. A second coating of the composition was then applied to the fingernail to remove a small amount of residual nail polish. The nail polish was completely removed from the fingernail 3 minutes and 40 seconds after the first application (FIG. 1(e)).

The above method was repeated for a fingernail to which gel nail polish was adhered. The time required for removal of the gel nail polish did not significantly differ to the time required to remove shellac.

### Example 2

The nail polish remover composition of Example 2 was prepared using the same procedure as described in Example 1.

Using the method described in Example 1, the nail polish remover composition of Example 2 was tested for removal of shellac nail polish from a fingernail. The total time required for removal of the shellac nail polish was 8 minutes 30 seconds.

### Example 3

In preparing the nail polish remover composition of Example 3, the ethanol caprylic/capric triglyceride and glycerine were combined and mixed until uniform. The hydroxypropyl cellulose (Klucel HF Pharm) was then sprinkled into the batch while mixing. The mixing was continued until the hydroxypropyl cellulose was completely dispersed and the batch composition thickened to form a gel.

Using the method described in Example 1, the nail polish remover composition of Example 3 was tested for removal of gel nail polish from a fingernail. The composition began dissolving the gel nail polish immediately upon application. A second coating of the composition was applied at 4 minutes and a third coating was applied at 6 minutes.

### Example 4

In preparing the nail polish remover composition of Example 4, the ethanol and propylene glycol monomethyl ether were combined and then mixed until uniform. The caprylic/capric triglyceride was then added and the combined components mixed until uniform. The hydroxypropyl cellulose (Klucel HF Pharm) was then sprinkled into the batch while mixing. The mixing was continued until the hydroxypropyl cellulose was completely dispersed and the batch composition thickened to form a gel.

Using the method described in Example 1, the nail polish remover composition of Example 4 was tested for removal of gel nail polish from a fingernail.

### Example 5

The nail polish remover composition of Example 5 was prepared using the same procedure as described in Example 4.

Using the method described in Example 1, the nail polish remover composition of Example 5 was tested for removal of gel nail polish from a fingernail. The composition began dissolving the gel nail polish immediately upon application. The total time required for removal of the shellac was 7 minutes 30 seconds.

The compositions, articles and methods described herein are presented by way of example only and are not limiting as to the scope of the invention. Unless otherwise specifically stated, individual aspects and components of the compositions, articles and methods may be modified, or may have been substituted therefore known equivalents, or as yet unknown substitutes such as may be developed in the future or such as may be found to be acceptable substitutes in the future. The compositions, articles and methods may also be modified for a variety of applications while remaining within the scope and spirit of the claimed invention, since the range of potential applications is great, and since it is intended that the present compositions, articles and methods be adaptable to many such variations.

1. A composition for removing nail polish from a surface, said composition comprising:
(i) a solvent, said nail polish being at least partially soluble in said solvent; and
(ii) a gelling agent,
wherein the gelling agent forms a gel matrix, said solvent being included within said gel matrix.

2. The composition of claim 1, wherein said solvent is acetone.

3. The composition of claim 1, wherein the concentration of said solvent in said composition is between 50 and 99% by weight of the total composition.

4. (canceled)

5. The composition of claim 1, to wherein the concentration of said gelling agent in said composition is between 1.5 and 10% by weight of the total composition.

6. The composition of claim 1, further comprising:
one or more emollients; or
(ii) one or more emollients are selected from the group consisting of cetyl alcohol, acetylated lanolin alcohol, caprylic/capric triglyceride, glycerine and combinations thereof.

7. (canceled)

8. The composition of claim 6, wherein the concentration of said one or more emollients in said composition is between 1 and 20% by weight of the total composition.

9. The composition of claim 1, further comprising a co-solvent.

10. The composition of claim 9, wherein said co-solvent is purified water or propylene glycol monomethyl ether.

11. The composition of claim 9, wherein the concentration of said co-solvent in said composition is between 1 and 45% by weight of the total composition.

12. The composition of claim 1, which is in the form of a weak gel.

13. The composition of claim 1, which is in the form of a strong gel.

14. The composition of claim 13, wherein the composition has a first surface adapted to substantially conform to a fingernail or toenail.

15. (canceled)

16. An article for removing nail polish from a fingernail or toenail, said article comprising:
(a) a substrate, said substrate having a first surface adapted to substantially conform to said fingernail or toenail; and
(b) the composition of claim 1;
wherein said composition is disposed on said first surface of said substrate such that said composition is configured to contact said fingernail or toenail.

17. The article of claim 16, wherein the size and shape of said first surface substantially corresponds to the size and shape of the fingernail or toenail.

18. The article of claim 16, wherein the first surface is approximately 1.5 cm wide and 2 cm long.

19. The article of claim 16, wherein the substrate is substantially impermeable to the composition.

20. The article of claim 16 further comprising a covering layer, said covering layer being adapted to at least substantially cover said composition, such that said composition forms a layer between said first surface and said covering layer, wherein said covering layer is substantially impermeable to said composition.

21. (canceled)

22. A method of removing nail polish from a surface, the method comprising the steps of:
(a) providing a composition of claim 1;
(b) contacting said composition with said nail polish;
(c) allowing sufficient time for said solvent to diffuse from said gel matrix to at least partially dissolve said nail polish; and
(d) removing said at least partially dissolved nail polish from said surface.

23. The method of claim 22, wherein said time in step (c) is less than 15 minutes, or between 1 minute and 5 minutes.

24. (canceled)

25. The method of claim 22, wherein said nail polish is shellac or gel nail polish.

26.-28. (canceled)
